(19) United States
(12) Patent Application Publication
(10) Pub. No.: US 2023/0068975 A1
(43) Pub. Date: Mar. 2, 2023

(54) IMAGING APPARATUS FOR ENDOSCOPE AND METHOD OF MANUFACTURING SAME

(71) Applicant: Sharp Semiconductor Innovation Corporation, Tenri City (JP)

(72) Inventors: NAKAE NAKAMURA, Tenri City (JP); Nobuaki ASAYAMA, Tenri City (JP); Tadahiko SATO, Tenri City (JP); Yan QIAN, Tenri City (JP)

(21) Appl. No.: 17/887,298

(22) Filed: Aug. 12, 2022

(30) Foreign Application Priority Data
Aug. 30, 2021 (JP) 2021-140345

(51) Int. Cl. A61B 1/00; H04N 5/225; A61B 1/07; A61B 1/05

(52) U.S. Cl. CPC A61B 1/00096; H04N 5/2256; H04N 5/2252; A61B 1/0011; A61B 1/07; A61B 1/05; H04N 2005/2255

(57) ABSTRACT

An imaging apparatus for an endoscope includes: a lens; an image sensor configured to capture an image via the lens; a light blocking portion provided covering the lens and the image sensor; a light source provided near the light blocking portion; and a light guide portion provided covering the light blocking portion and the light source and configured to guide light from the light source. The light blocking portion is provided so as to prevent the light from the light source from being incident on the lens and the image sensor. An end face of the lens is arranged at a position further recessed than at least an end face of the light blocking portion.

### CROSS-REFERENCE TO RELATED APPLICATION

The present application claims priority from Japanese Patent Application Number 2021-140345, the content to which is hereby incorporated by reference into this application.

### BACKGROUND OF THE INVENTION

### 1. Field of the Invention

The disclosure relates to an imaging apparatus for an endoscope and a method of manufacturing the same.

### 2. Description of the Related Art

In the related art, an endoscope system including an image sensor and a light source for the purpose of imaging inside the body has been widely used. In particular, in an endoscope used for observing a narrow portion of the body such as inside a blood vessel, a compact module in which the image sensor and the light source are integrated in a resin or the like is used. The disclosure disclosed in JP 2018-180185 A is a related technology.

JP 2018-180185 A relates to a compact imaging apparatus with a light for medical use or industrial use. In the compact imaging apparatus provided in the compact imaging apparatus with a light, a light end is disposed in a gap between a camera body or an image guide and an outer peripheral protective tube, the light end is a molded body made of a transparent synthetic resin material formed to match the shape of the gap, a light transmission member is connected to a rear portion of the light end, and illumination light emitted from a light source is transmitted from an incident end at a rear end of the light transmission member to the light end.

In the compact imaging apparatus with a light disclosed in JP 2018-180185 A, an end face of a lens disposed on a front face of the camera body is curved into a convex shape and protrudes from the transparent synthetic resin material. As a result, the lens end face may become damaged or dirty due to contact with an instrument or the like at the time of manufacture or use, thereby degrading image quality.

Further, in the method of manufacturing a compact imaging apparatus with a light described in JP 2018-180185 A, the transparent synthetic resin material may adhere to the lens end face, or the lens end face may become damaged due to contact with a tool used for dispensing the resin.

An object of an aspect of the disclosure is to provide an imaging apparatus for an endoscope that prevents a lens end face from being damaged or dirtied, and a method of manufacturing the same.

### SUMMARY OF THE INVENTION

To address the above problem, an imaging apparatus for an endoscope according to an aspect of the disclosure includes: a lens, an image sensor configured to capture an image via the lens, a light blocking portion provided covering the lens and the image sensor, a light source provided near the light blocking portion, and a light guide portion provided covering the light blocking portion and the light source and configured to guide light from the light source, in which the light blocking portion is provided so as to prevent the light from the light source from being incident on the lens and the image sensor, and an end face of the lens is arranged at a position further recessed than at least an end face of the light blocking portion.

To address the above problem, a method of manufacturing an imaging apparatus for an endoscope according to an aspect of the disclosure includes: attaching a light blocking portion so as to cover a lens and an image sensor configured to capture an image via the lens, forming an outer frame surrounding the light blocking portion, injecting a resin into the outer frame, inserting a light source into the resin injected between the outer frame and the light blocking portion, and curing the resin to form a light guide portion, in which an end face of the lens is arranged at a position more recessed than at least an end face of the light blocking portion.

To address the above problem, a method of manufacturing an imaging apparatus for an endoscope according to an aspect of the disclosure includes: attaching a light blocking portion so as to cover a lens and an image sensor configured to capture an image via the lens, fixing and positioning the fight blocking portion and a light source to an outer frame member configured to surround the blocking portion, forming the outer frame, injecting a resin into the outer frame, curing the resin to form a light guide portion, in which an end face of the lens is arranged at a position more recessed than at least an end face of the light blocking portion.

An aspect of the disclosure provides an imaging apparatus for an endoscope and a method of manufacturing the same that prevent a lens end face from being damaged or dirtied.

### BRIEF DESCRIPTION OF THE DRAWINGS

FIG. 1 is a perspective view of an imaging apparatus for an endoscope according to a first embodiment of the disclosure.

FIG. 2 is a perspective view when a light guide portion and an outer frame are removed from the imaging apparatus for an endoscope according to the first embodiment of the disclosure.

FIG. 3 is a cross-sectional view of the imaging apparatus for an endoscope according to the first embodiment of the disclosure.

FIG. 4 is a flowchart showing a method of manufacturing the imaging apparatus for an endoscope according to the first embodiment of the disclosure.

FIG. 5 is a diagram (1) illustrating a light blocking material being applied to a lens and an image sensor.

FIG. 6 is a diagram (2) illustrating the light blocking material being applied to the lens and the image sensor.

FIG. 7 is a diagram (3) illustrating the light blocking material being applied to the lens and the image sensor.

FIG. 8 is a diagram illustrating a method of forming the outer frame.

FIG. 9 is a diagram illustrating another method of forming the outer frame.

FIG. 10 is a diagram (1) illustrating injection of a resin into the outer frame.

FIG. 11 is a diagram (2) illustrating injection of the resin into the outer frame.

FIG. 12 is a diagram illustrating insert on of components into the outer frame.

FIG. 13 is a diagram illustrating curing of a UV-curing resin by irradiating the UV-curing resin with UV.

FIG. 14 is a diagram illustrating a process of forming the outer frame according to a second embodiment of the disclosure.

FIG. 15 is a diagram illustrating another example of the method of forming the outer frame according to the second embodiment of the disclosure.

FIG. 16 is a top view illustrating the shape of an outer frame according to a third embodiment of the disclosure.

FIG. 17 is a diagram illustrating a method of forming the outer frame according to the third embodiment of the disclosure.

FIG. 18 is a perspective view of an imaging apparatus for an endoscope according to a fourth embodiment of the disclosure.

FIG. 19 is a perspective view when a light guide portion is removed from the imaging apparatus for an endoscope according to the fourth embodiment of the disclosure.

FIG. 20 is a cross-sectional view of the imaging apparatus for an endoscope according to the fourth embodiment of the disclosure.

### DETAILED DESCRIPTION OF THE INVENTION

### First Embodiment

An embodiment of the disclosure will be described in detail below. Note that for the sake of convenience of description, identical members are given the same reference signs, and the names and functions thereof are the same. Thus, detailed descriptions will not be repeated for the components.

### Configuration Example of imaging Apparatus 1 for Endoscope

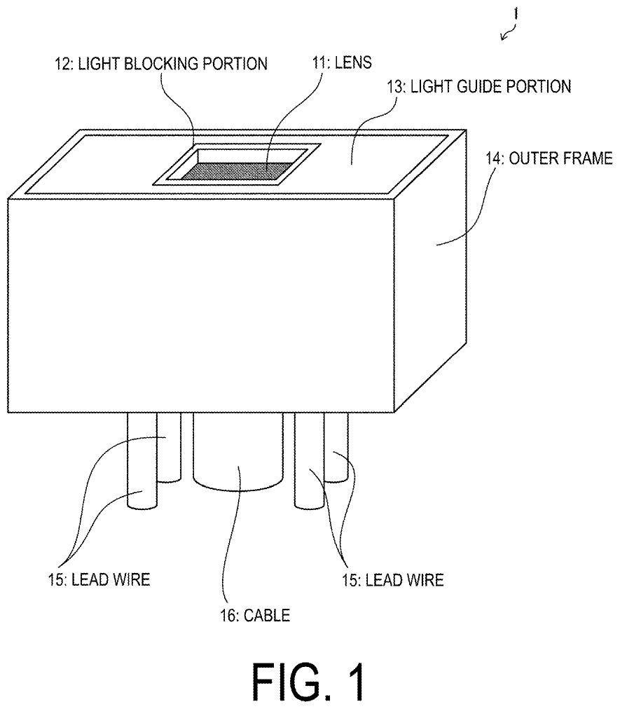

FIG. 1 is a perspective view of an imaging apparatus 1 for an endoscope according to a first embodiment of the disclosure. As illustrated in FIG. 1, the external shape of the imaging apparatus 1 for an endoscope is quadrilateral and is, for example, a rectangle. The external shape of the imaging apparatus for an endoscope is not limited to a rectangle and may be a square or another shape.

The area around a lens 11 is covered by a light blocking portion 12 that blocks light from a night source. The area around the light blocking portion 12 is covered by a light guide portion 13 that guides the light from the light source. The light guide portion 13 formed of a transparent material such as a transparent resin Further, the area around the light guide portion 13 is covered by an outer frame 14. The outer frame 14 is formed of a member that poses no danger to the human body. The outer frame 14 may also be formed using an opaque material, or may be formed using a transparent material so that the light from the light source is also irradiated in a side surface direction.

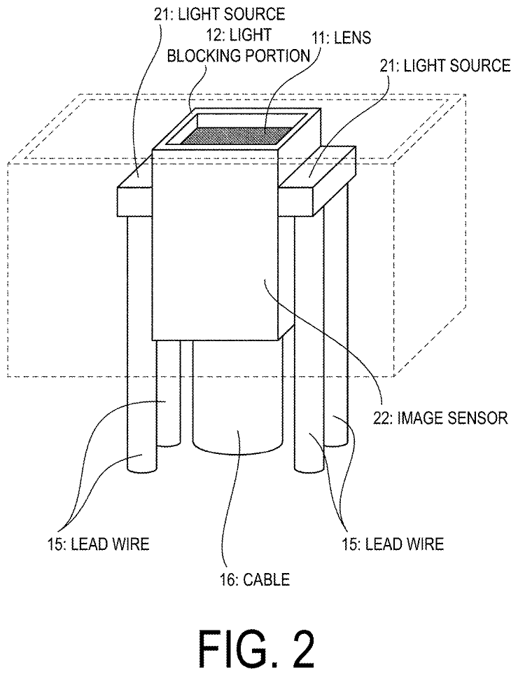

FIG. 2 is a perspective view in a case where the light guide portion 13 and the outer frame 14 are removed from the imaging apparatus 1 for an endoscope according to the first embodiment of the disclosure. Two light sources 21 are arranged around the light blocking portion 12. The light source 21 is configured of a light-emitting diode (LED). A lead wire 15 for power supply is connected to each of the light sources 21. Although FIG. 2 illustrates a case where the number of light sources 21 is two, the number of light sources 21 is not limited to two and may be one, or three or more.

The lens 11 and an image sensor 22 are provided inside the light blocking portion 12. A cable 16 for inputting/outputting a signal or the like is connected to the image sensor 22 provided inside the light blocking portion 12. The image sensor 22 captures an image via the lens 11 and outputs the captured image data to an external device via the cable 16.

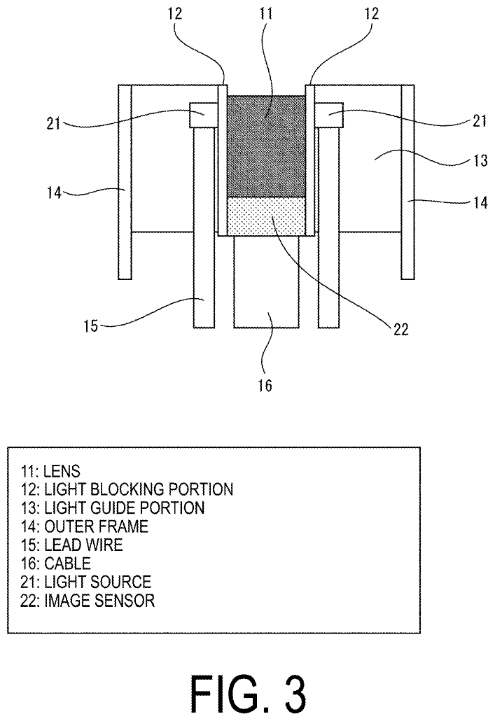

FIG. 3 is a cross-sectional view of the imaging apparatus 1 for an endoscope according to the first embodiment of the disclosure. The lens 11 and the image sensor 22 are provided inside the light blocking portion 12 in contact with each other. The cable 16 is connected to the image sensor 22. As illustrated in FIG. 3, an end face of the lens 11 is arranged at a lower position (recessed position) than an end face of the light blocking portion 12 and an end face of the light guide portion 13 in the vicinity of the lens 11. Note that, the number of lenses 11 may be one or more.

By arranging the end face of the lens 11 at a lower position (recessed position) than the end face of the light blocking portion 12 and the end face of the light guide portion 13 in the vicinity of the lens 11, the end face of the lens 11 can be prevented from being damaged or dirtied by coming into contact with an instrument or the like at the time of manufacturing or use.

### Method of Manufacturing Imaging Apparatus 1 for Endoscope

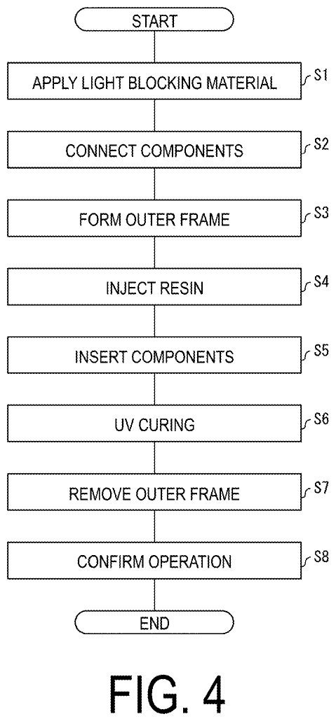

FIG. 4 is a flowchart showing a method of manufacturing the imaging apparatus 1 for an endoscope according to the first embodiment of the disclosure. First, the light blocking portion 12 is attached to the lens 11 and the image sensor 22 (S1).

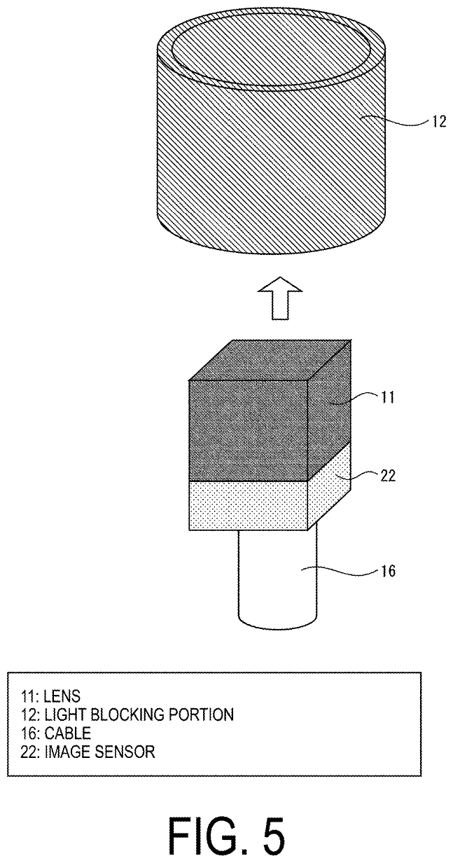

FIGS. 5 to 7 are diagrams illustrating a light blocking material being applied to the lens 11 and the image sensor 22. As illustrated in FIG. 5, the lens 11 and the image sensor 22 are inserted into the light blocking portion 12. The light blocking portion 12 is formed of a light blocking material. Hereinafter, a case where a heat shrink tube is used as an example of the light blocking material constituting the light blocking portion 12 will be described.

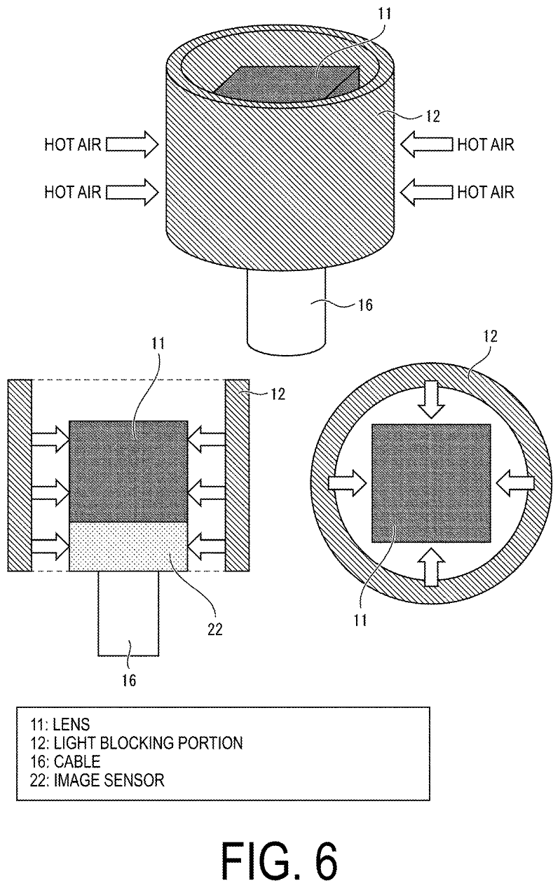

Subsequently, as illustrated in the perspective view at the top of FIG. 6, hot air is applied to the light blocking portion 12 to heat the light blocking portion 12 while the lens 11 and the image sensor 22 are inserted into the light blocking portion 12. As illustrated in the cross-sectional view at the bottom left of FIG. 6, the light blocking portion 12 shrinks in the direction indicated by the arrow when heat is applied. The top view at the lower right of FIG. 6 is a top view illustrating an example of how the light blocking portion 12 shrinks.

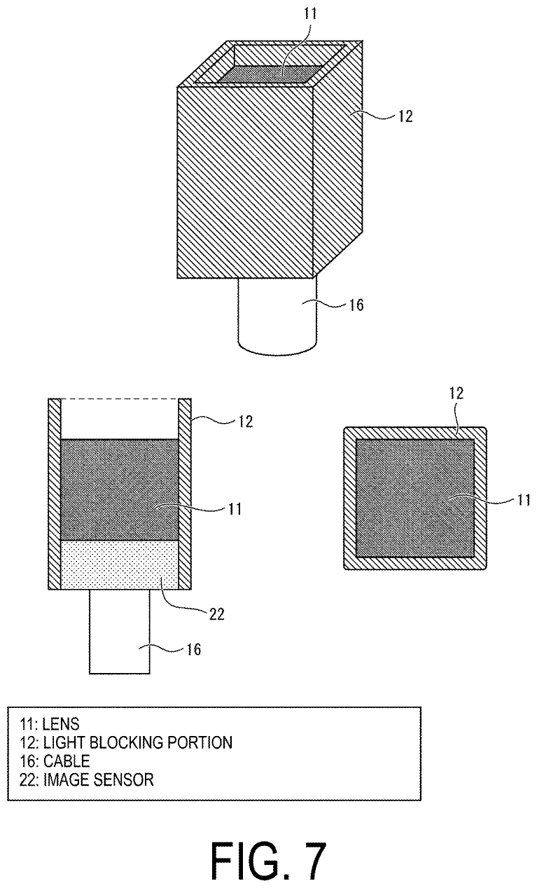

As illustrated in the perspective view at the top of FIG. 7, the light blocking portion 12 shrinks along the external shape of the lens 11 and the image sensor 22, and thus the light blocking portion 12 is formed. As illustrated in the cross-sectional view at the lower left of FIG. 7, the light blocking portion 12 is formed such that the end face of the lens 11 is arranged at a lower position (recessed position) than the end face of the light blocking portion 12 in the vicinity of the lens 11. The top view at the lower right of FIG. 7 is a top view of the lens 11 and the light blocking portion 12.

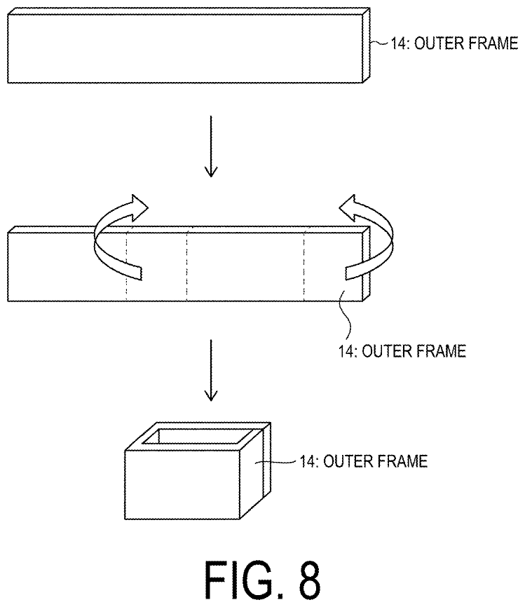

Subsequently, as shown in the flowchart of FIG. 4, the components are connected (S2). For example, the light sources 21 and the lead wires 15 illustrated in FIG. 2 are connected. Subsequently, the outer frame 14 surrounding the light blocking portion 12 is formed (S3). FIG. 8 is a diagram illustrating a method of forming the outer frame 14. The outer frame 14 is composed of a sheet member or the like that poses no danger to the human body. As illustrated in FIG. 8, the outer frame 14 is folded at dotted line portions (fold line portions) to form the outer frame 14 having a rectangular shape.

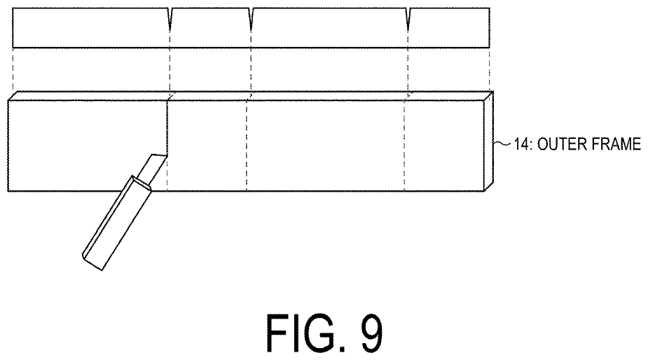

FIG. 9 is a diagram illustrating another method of forming the outer frame 14. As illustrated in FIG. 9, the outer frame 14 is cut in advance by using a utility knife or the like at the fold line portions and is folded along the fold line portions. The depth of each cut is set to about ⅓ to ½ of the thickness of the sheet member so that the sheet member is not fully cut away.

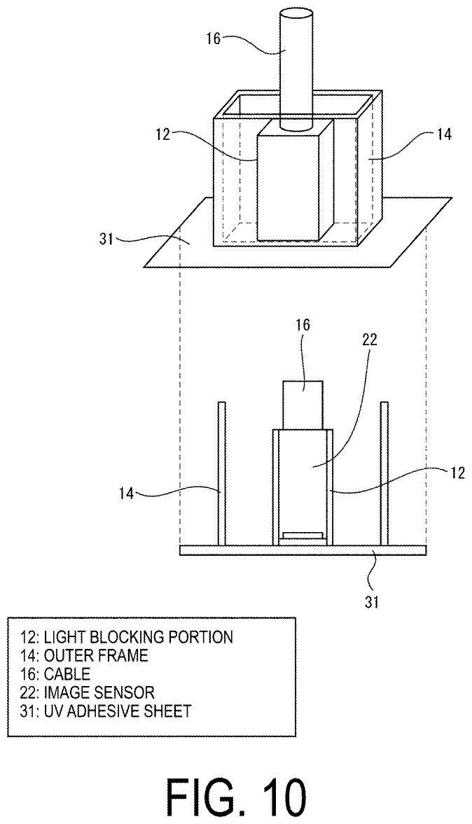

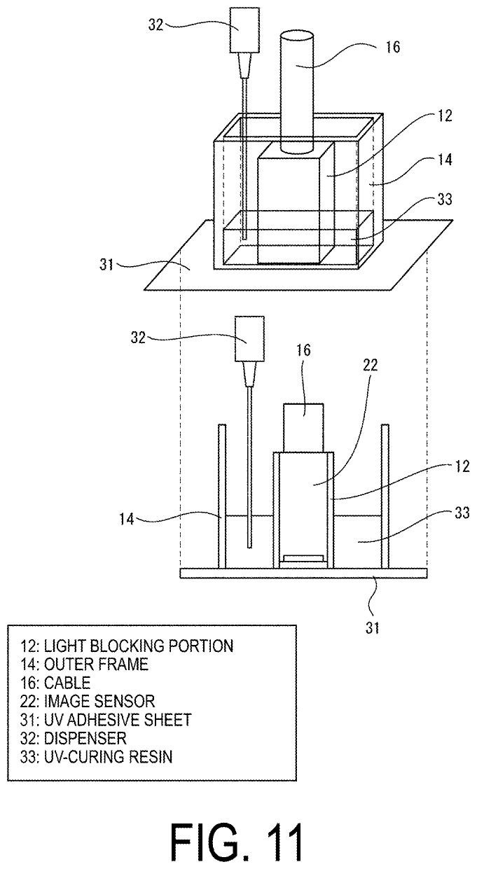

Subsequently, as shown in the flowchart of FIG. 4, a resin is injected into the outer frame 14 (S4). The resin is, for example, a transparent ultraviolet (UV)-curing resin. FIGS. 10 and 11 are diagrams illustrating injection of the resin into the outer frame 14.

As illustrated in the perspective view at the top of FIG. 10, the outer frame 14 is placed on a UV adhesive sheet 31, the light blocking portion 12 is arranged with the end face of the lens 11 at the bottom, and the outer frame 14 and the light blocking portion 12 are fixed to the UV adhesive sheet 31. As illustrated in the cross-sectional view at the bottom of FIG. 10, the light blocking portion 12 covering the image sensor 22 is fixed at the substantial center of the outer frame 14.

As illustrated in the perspective view at the top of FIG. 11, a UV-curing resin 33 is injected into the outer frame 14 by a dispenser 32. As illustrated in the cross-sectional view at the bottom of FIG. 11, the UV-curing resin 33 does not leak to the outside because the outer frame 14 is fixed to the UV adhesive sheet 31. In addition, the UV-curing resin 33 does not adhere to the end race of the lens 11 because the light blocking portion 12 is also fixed to the UV adhesive sheet 31.

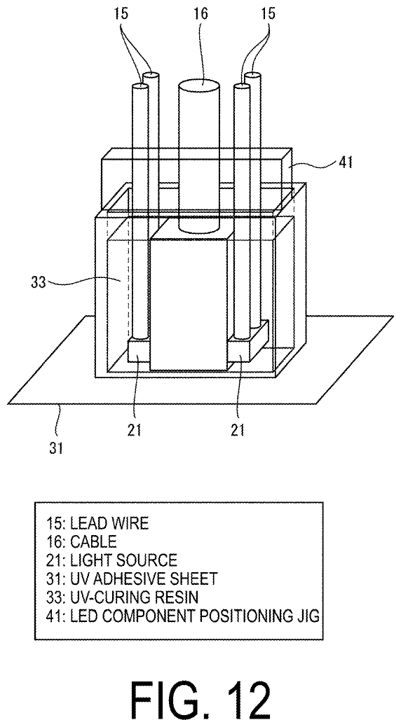

Subsequently, as shown in the flowchart of FIG. 4, components are inserted into the outer frame 14 (S5). The components are the light sources 21, the lead wires 15 connected to the light sources 21, and the like. FIG. 12 is a diagram illustrating the components being inserted into the outer frame 14. As illustrated in FIG. 12, the light sources 21 and the lead wires 15 are inserted into the UV-curing resin 33, which has been injected into the outer frame 14. At this time, the depth of insertion is adjusted by an LED component positioning jig 41 so that each light source 21 is located at a predetermined position.

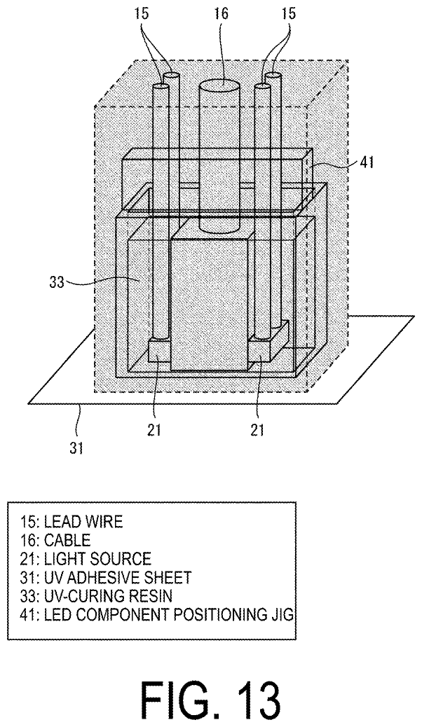

Subsequently, as shown in the flowchart of FIG. 4, the UV-curing resin 33 is cured by irradiating the UV-curing resin 33 with UV (S6). FIG. 13 is a diagram illustrating curing of the UV-curing resin 33 by irradiating the UV-curing resin 33 with UV.

As illustrated in FIG. 13, the UV-curing resin 33 is cured by irradiating the entire UV-curing resin 33 with UV. At this time, since the UV adhesive sheet 31 is also irradiated. with UV, the adhesive force of the UV adhesive sheet 31 decreases. Because the adhesive force of the UV adhesive sheet 31 decreases, the UV adhesive sheet 31 can be easily removed. Note that, curing the UV-curing resin 33 forms the light guide portion 13.

Subsequently, as shown in the flowchart of FIG. 4, in a case where it is necessary to remove the outer frame 14, the outer frame 14 is removed from the light guide portion 13 (S7). In the present embodiment, there is no need to remove the outer frame 14 so this process is not performed. Lastly, the operation of the assembled imaging apparatus 1 for an endoscope is checked by using a conduction checker or the like (S8), and the process ends.

### Effects of Imaging Apparatus 1 for an Endoscope

As described above, in the imaging apparatus 1 for an endoscope according to the present embodiment, because the end face of the lens 11 is arranged at a lower position (recessed position) than the end face of the light blocking portion 12 and the end face of the light guide portion 13 in the vicinity of the lens 11, the end face of the lens 11 can be prevented from being damaged or dirtied due to contact with an instrument or the like at the time of manufacture or use.

With the lens 11 and the image sensor 22 inserted into the light blocking portion 12, hot air is applied to the light blocking portion 12 to form the light blocking portion 12. Therefore, the light blocking portion 12 can be easily formed along the external shape of the lens 11 and the image sensor 22.

In addition, since the outer frame 14 is formed by folding a sheet-shaped member, there is no need to use a metal material or a casting mold, reducing costs.

Further, the UV-curing resin 33 does not leak to the outside because the outer frame 14 is fixed to the UV adhesive sheet 31. In addition, the UV-curing resin 33 does not adhere to the end face of the lens 11 because the light blocking portion 12 is also fixed to the UV adhesive sheet 31.

Since positioning is performed while the light sources 21 and the lead wires 15 are inserted into the UV-curing resin 33 injected into the outer frame 14 (state before the UV-curing resin 33 is cured), position adjustment of the light sources 21 and other components can be easily performed. Further, appearance defects can be easily confirmed and easily repaired.

### Second Embodiment

A second embodiment of the disclosure will be described in detail below. Note that for the sake of convenience of description, identical members are given the same reference signs, and the names and functions thereof are the same. Thus, detailed descriptions will not be repeated for the components.

In the first embodiment of the disclosure, after the UV-curing resin 33 is injected into the outer frame 14, the light sources 21 and other components are inserted into the UV-curing resin 33 and positioned. In the second embodiment of the disclosure, the light sources 21 and other components are positioned when the outer frame 14 is formed.

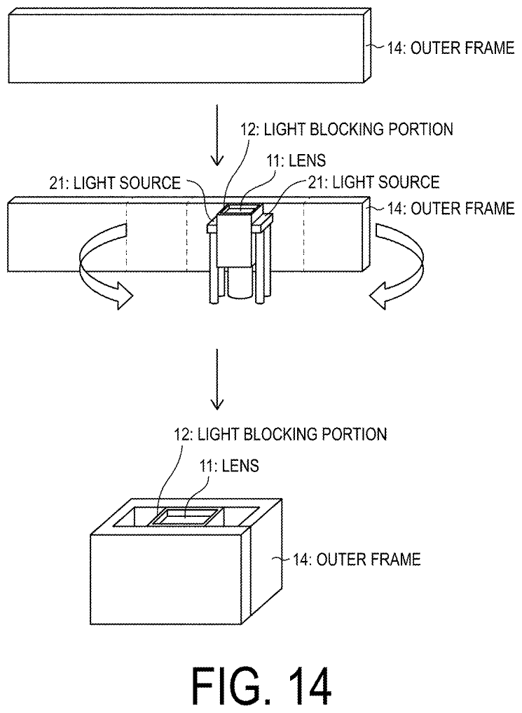

FIG. 14 is a diagram illustrating a process of forming the outer frame (S3) according to the second embodiment of the disclosure. In the present embodiment, since she light sources 21 and other components are positioned when forming the outer frame, the process of inserting the components (S5) is not required.

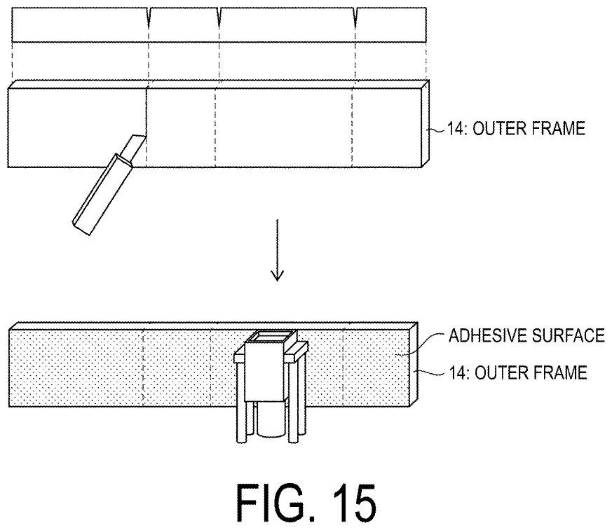

As illustrated in FIG. 14, the member of the outer frame 14, for example, a strip-shaped sheet member is folded, and the light blocking portion 12 and the light sources 21 are fixed to the sheet member and positioned. The sheet member has an adhesive surface as illustrated in FIG. 15. The light blocking portion 12 and the light sources 21 are fixed to the adhesive surface. Then, the sheet member is folded along the fold lines to complete the outer frame 14. At this time, for injecting the UV-curing resin 33, a gap is preferably left in a portion opposite a portion of the outer frame 14 on the side where the light blocking portion 12 and the light sources 21 are fixed.

FIG. 15 is a diagram illustrating another example of the method of forming the outer frame 14 according to the second embodiment of the disclosure. As illustrated in FIG. 15, the outer frame 14 is cut in advance by using a utility knife or the like at the fold line portions and is folded along the fold line portions. The depth of each cut is set to about ⅓ to ½ of the thickness of the sheet member so that the sheet member is not fully cut away. A sheet member having adhesiveness may be used as the sheet member for forming the outer frame 14, and the light blocking portion 12 and the light sources 21 may be fixed to an adhesive surface of the sheet member.

As described above, in the method of manufacturing the imaging apparatus 1 for an endoscope according to the present embodiment, since the UV-curing resin 33 is injected after the light blocking portion 12 and the light sources 21 are positioned at the time of forming the outer frame 14, the procedure of inserting and positioning the light sources 21 and other components into the UV-curing resin 33 can be omitted.

### Third Embodiment

A third embodiment of the disclosure will be described in detail below. Note that for the sake of convenience of description, identical members are given the same reference signs, and the names and functions thereof are the same. Thus, detailed descriptions will not be repeated for the components.

In the first embodiment of the disclosure, the outer frame 14 has a rectangular shape, and two light sources 21 are provided. In the third embodiment of the disclosure, three light sources 21 are provided, and the shape of the outer frame 14 is different to a rectangle.

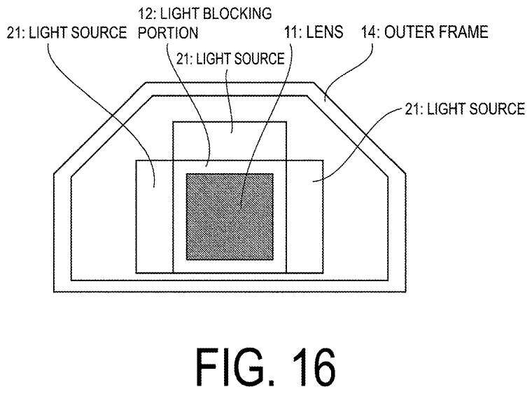

FIG. 16 is a top view illustrating the shape of the outer frame 14 according to the third embodiment of the disclosure. As illustrated in FIG. 16, three light sources 21 are provided around the light blocking portion 12. Therefore, the outer frame 14 is formed by folding a strip-shaped sheet member into a hexagon.

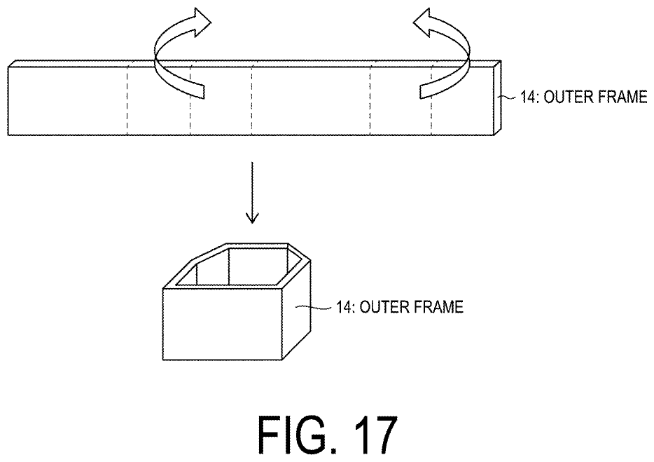

FIG. 17 is a diagram illustrating a method of forming the outer frame 14 according to the third embodiment of the disclosure. As illustrated in FIG. 17, the outer frame 14 is folded at dotted line portions (fold line portions) to form the outer frame 14 having a hexagonal shape.

As described above, in the imaging apparatus for an endoscope according to the present embodiment, for example, three light sources 21 are provided and the shape of the outer frame 14 is made to be a shape different from a rectangle. Since a strip-shaped sheet member whose shape can be easily changed is used as the outer frame 14, the external shape of the imaging apparatus for an endoscope can be easily changed according to changes to the constituent elements of the imaging apparatus for an endoscope and the intended usage.

### Fourth Embodiment

A fourth embodiment of the disclosure will be described in detail below. Note that for the sake of convenience of description, identical members are given the same reference signs, and the names and functions thereof are the same. Thus, detailed descriptions will not be repeated for the components.

In the first embodiment of the disclosure, the outer frame 14 is formed of a member that poses no danger to the human body, and the outer frame 14 is not removed. The present embodiment includes a step of removing the outer frame 14 from the imaging apparatus 1 for an endoscope (S7 in FIG. 4).

### Configuration Example of Imaging Apparatus 1A for Endoscope

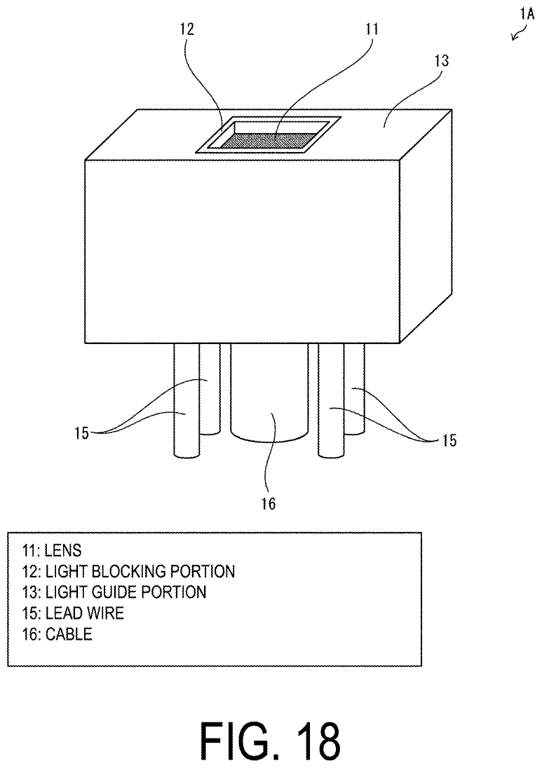

FIG. 18 is a perspective view of an imaging apparatus 1A for an endoscope according to the fourth embodiment of the disclosure. As illustrated in FIG. 18, the external shape of the imaging apparatus 1A for an endoscope is quadrilateral and is, for example, a rectangle. Note that the external shape of the imaging apparatus 1A for an endoscope is not limited to a rectangle, and may be a square or another shape. The imaging apparatus 1A for an endoscope differs from the imaging apparatus 1 for an endoscope according to the first embodiment illustrated in. FIG. 1 in that the outer frame 14 is removed.

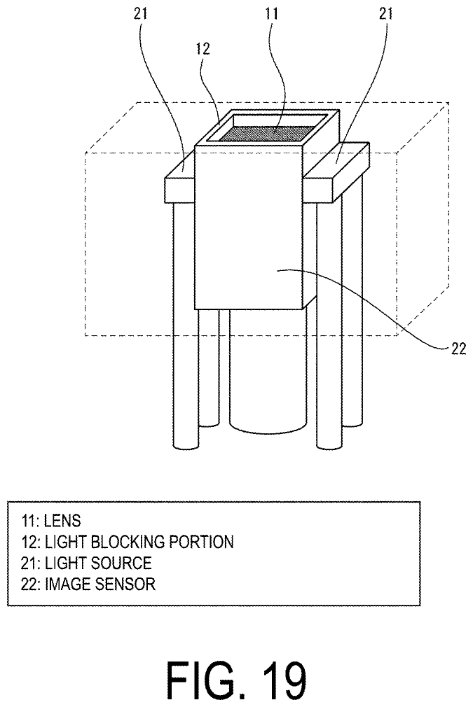

FIG. 19 is a perspective view illustrating a case where the light guide portion 13 is removed from the imaging apparatus 1A for an endoscope according to the fourth embodiment of the disclosure. Two light sources 21 are arranged around the light blocking portion 12. The lead wire 15 for power supply is connected to each of the light sources 21. Although FIG. 19 illustrates a case where the number of light sources 21 is two, the number of light sources 21 is not limited to two, and may be one, or three or more.

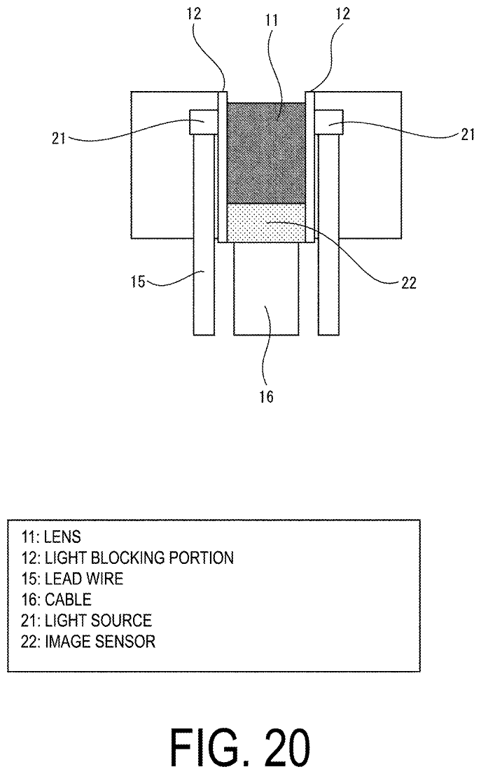

FIG. 20 is a cross-sectional view of the imaging apparatus 1A for an endoscope according to the fourth embodiment of the disclosure. The lens 11 and the image sensor 22 are provided inside the light blocking portion 12 in contact with each other. The cable 16 is connected to the image sensor 22. As illustrated in FIG. 20, the end face of the lens 11 is arranged at a lower position (recessed position) than the end face of the light blocking portion 12 and the end face of the light guide portion 13 in the vicinity of the lens 11. Note that, the number of lenses 11 may be one or more.

As described above, in the imaging apparatus 1A for an endoscope according to the present embodiment, the end face of the lens 11 is arranged at a lower position (recessed position) than the end face of the light blocking portion 12 and the end face of the light guide portion 13 the vicinity of the lens 11. As a result, the end face of the lens 11 can be prevented from being damaged or dirtied due to contact with an instrument or the like at the time of manufacture or use.

Further, in the present embodiment, since the outer frame is removed, it is not necessary to form the outer frame of a member that poses no danger to the human body. Therefore, the outer frame can be formed of a more inexpensive material, and costs at the time of manufacturing the imaging apparatus 1A for an endoscope can be reduced.

### Supplement

An imaging apparatus for an endoscope according to a first aspect of the disclosure includes:

a lens;

an image sensor configured to capture an image via the lens;

a light blocking portion provided covering the lens and the image sensor;

a light source provided near the light blocking portion; and

a light guide portion provided covering the light blocking portion and the light source and configured to guide light from the light source,

in which the light blocking portion is provided so as to prevent the light from the light source from being incident on the lens and the image sensor, and

an end face of the lens is arranged at a posit on further recessed than at least an end face of the light blocking portion.

According to this configuration, the end face of the lens can be prevented from being damaged or dirtied due to contact with an instrument or the like at the time of manufacture or use.

An imaging apparatus for an endoscope according to a second aspect of the disclosure is the imaging apparatus for an endoscope according to the first aspect, further including an outer frame provided covering the light guide portion.

According to this configuration, since the outer frame is formed by folding a sheet-shaped member it is not necessary to use a metal material or a casting mold, making it possible to reduce costs.

An imaging apparatus for an endoscope according to a third aspect of the disclosure is the imaging apparatus for an endoscope according to the first or second aspect, in which the end face of the lens is arranged at a position more recessed than at least the end face of the light blocking portion and an end face of the light guide portion.

According to this configuration, the end face of the lens can be prevented from being damaged or dirtied due to contact with an instrument or the like at the time of manufacture or use.

An imaging apparatus for an endoscope according to a fourth aspect of the disclosure is the imaging apparatus for an endoscope according to any one of the first to third aspects, in which the light blocking portion is a heat shrink tube.

According to this configuration, the light blocking portion can be easily formed along the external shape of the lens and the image sensor.

An imaging apparatus for an endoscope according to a fifth aspect of the disclosure is the imaging apparatus for an endoscope according to any one of the first to fourth aspects, in which the light guide portion is a UV-curing resin.

According to this configuration, the position of the light source or the like can be easily adjusted. Further, appearance defects can be easily confirmed and easily repaired.

A method of manufacturing an imaging apparatus for an endoscope according to a sixth aspect of the disclosure includes:

attaching a blocking portion so as to cover a lens and an image sensor configured to capture an image via the lens;

forming an outer frame surrounding the light blocking portion;

injecting resin into the outer frame;

inserting a light source into the resin injected between the outer frame and the light blocking portion; and

curing the resin to form a light guide portion,

wherein an end face of the lens is arranged at a position more recessed than at least an end face of the light blocking portion.

According to this configuration, the end face of the lens can be prevented from being damaged or dirtied due to contact with an instrument or the like at the time of manufacture or use.

A method of manufacturing an imaging apparatus for an endoscope according to a seventh aspect of the disclosure includes:

attaching a light blocking portion so as to cover a lens and an image sensor configured to capture an image via the lens;

fixing and positioning the light blocking portion and a light source to an outer frame member configured to surround the light blocking portion;

forming the outer frame;

injecting a resin into the outer frame; and

curing the resin to form a light guide portion,

wherein an end face of the lens is arranged at a position more recessed than at least an end face of the light blocking portion.

A method or manufacturing an imaging apparatus for an endoscope according to an eighth aspect of the disclosure is the method of manufacturing an imaging apparatus for an endoscope according to the sixth or seventh aspect, in which the method of manufacturing further includes removing the outer frame.

According to this configuration, the outer frame can be configured of a less expensive material, making it possible to reduce costs at the time of manufacturing the imaging apparatus for an endoscope.

A method of manufacturing an imaging apparatus an endoscope according to a ninth aspect of the disclosure is the method of manufacturing an imaging apparatus for an endoscope according to any one of the sixth to eighth aspects, in which the end face of the lens is disposed at a position more recessed than at least the end race of the light blocking portion and an end face of the light guide portion.

According to this configuration, the end face of the lens can be prevented from being damaged or dirtied due to contact with an instrument or the like at the time of manufacture or use.

A method of manufacturing an imaging apparatus for an endoscope according to a tenth aspect of the disclosure is the method of manufacturing an imaging apparatus for an endoscope according to any one of the sixth to ninth aspects, in which attaching the light blocking portion includes inserting the lens and the image sensor into a heat shrink tube, and heating the heat shrink tube to form the light blocking portion.

According to this configuration, the light blocking portion can be easily formed along, the external shape of the lens and the image sensor.

A method of manufacturing an imaging apparatus for an endoscope according to an eleventh aspect of the disclosure is the method of manufacturing an imaging apparatus for an endoscope according to any one of the sixth to tenth aspects, in which injecting the resin in the outer frame includes injecting the resin in a state where the end face of the light blocking portion and a first end face of the outer frame are disposed on the same plane.

A method of manufacturing an imaging apparatus for an endoscope according to a twelfth aspect of the disclosure is the method of manufacturing an imaging apparatus for an endoscope according to the eleventh aspect, in which the end face of the light blocking portion and the first end face of the outer frame are disposed on the same plane by affixing the end face of the light blocking portion and the first end face of the outer frame to an adhesive sheet.

A method of manufacturing an imaging apparatus for an endoscope according to a thirteenth aspect of the disclosure is the method of manufacturing an imaging apparatus for an endoscope according to any one of the sixth to twelfth aspects, in which injecting the resin into the outer frame includes injecting a UV-curing resin into the outer frame, and curing the resin includes curing the UV-curing resin by irradiating the UV-curing resin with UV.

According to this configuration, the position of the light source or the like can be easily adjusted. Further, appearance defects can be easily confirmed and easily repaired.

The disclosure is not limited to each of the above-described embodiments. Various modifications are possible within the scope of the claims. An embodiment obtained by appropriately combining technical elements each disclosed in different embodiments falls also within the technical scope of the disclosure. Furthermore, technical elements disclosed in the respective embodiments may be combined to provide a new technical feature.

While there have been described what are at present considered to be certain embodiments of the invention, it will be understood that various modifications may be made thereto, and it is intended that the appended claims cover all such modifications as fall within the true spirit and scope of the invention.

What is claimed is:

1. An imaging apparatus for an endoscope comprising:
a lens;
an image sensor configured to capture an image via the lens;
a light blocking portion provided covering the lens and the image sensor;
a light source provided near the light blocking portion; and
a light guide portion provided covering the light blocking portion and the light source and configured to guide light from the light source,
wherein the light blocking portion is provided so as to prevent the light from the light source from being incident on the lens and the image sensor, and
an end face of the lens is arranged at a position further recessed than at least an end face of the light blocking portion.

2. The imaging apparatus for an endoscope according to claim 1, further comprising:
an outer frame provided covering the light guide portion.

3. The imaging apparatus for an endoscope according to claim 1,
wherein the end face of the lens is arranged at a position more recessed than at least the end face of the light blocking portion and an end face of the light guide portion.

4. The imaging apparatus for an endoscope according to claim 1,
wherein the light blocking portion is a heat shrink tube.

5. The imaging apparatus for an endoscope according to claim 1,
wherein the light guide portion is a UV-curing resin.

6. A method of manufacturing an imaging apparatus for an endoscope, the method comprising:
attaching a light blocking portion so as to cover a lens and an image sensor configured to capture an image via the lens;
forming an outer frame surrounding the light blocking portion;
injecting a resin into the outer frame;
inserting a light source into the resin injected between the outer frame and the light blocking portion; and
curing the resin to form a light guide portion,
wherein an end face of the lens is arranged at a position more recessed than at least an end face of the light blocking portion.

7. The method of manufacturing an imaging apparatus for an endoscope according to claim 6, further comprising:
removing the outer frame.

8. The method of manufacturing an imaging apparatus for an endoscope according to claim 6,
wherein the end face of the lens is arranged at a position more recessed than at least the end face of the light blocking portion and as end face of the light guide portion.

9. The method of manufacturing an imaging apparatus for an endoscope according to claim 6,
wherein attaching the light blocking portion includes
inserting the lens and the image sensor into a heat shrink tube, and
heating the heat shrink tube to form the light blocking portion.

10. The method of manufacturing an imaging apparatus for an endoscope according to claim 6,
wherein injecting the resin into the outer frame includes
injecting the resin in a state where the end face of the light blocking portion and a first end face of the outer frame are disposed on the same plane.

11. The method of manufacturing an imaging apparatus for an endoscope according to claim 10,
wherein the end face of the light blocking portion and the first end face of the outer frame are disposed on the same plane by affixing the end face of the light blocking portion and the first end face of the outer frame to an adhesive sheet.

12. The method of manufacturing an imaging apparatus for an endoscope according to claim 6,
wherein injecting the resin into the outer frame includes injecting a UV-curing resin into the outer frame, and
curing the resin includes curing the UV-curing resin by ng the UV-curing resin with UV.

13. A method of manufacturing an imaging apparatus for an endoscope, the method comprising:
attaching a light blocking portion so as to cover a lens and an image sensor configured to capture an image via the lens;
fixing and positioning the 1 g blocking portion and a light source to an outer frame member configured to surround the light blocking portion;
forming the outer frame;
injecting resin into the outer frame; and
curing the resin to form a light guide portion,
wherein an end face of the lens is arranged at a position more recessed than at least an end face of the light blocking portion.

14. The method of manufacturing an imaging apparatus for an endoscope according to claim 13, further comprising:
removing the outer frame.

15. The method of manufacturing as imaging apparatus for an endoscope according to claim 13,
wherein the end face of the lens is arranged at a position more recessed than at least the end face of the light blocking portion and an end face of the light guide portion.

16. The method of manufacturing as imaging apparatus for an endoscope according to claim 13,
wherein attaching the light blocking portion includes
inserting the lens and the image sensor into a heat shrink tube, and
heating the heat shrink tube to form the light blocking portion.

17. The method of manufacturing an imaging apparatus for an endoscope according to claim 13,
wherein injecting the resin into the outer frame includes injecting the resin in a state where the end face of the light blocking portion and a first end face of the outer frame are disposed on the same plane.

18. The method of manufacturing an imaging apparatus for an endoscope according to claim 17,
wherein the end face of the light blocking portion and the first end face of the outer frame are disposed on the same plane by affixing the end face of the light blocking portion and the first end face of the outer frame to an adhesive sheet.

19. The method of manufacturing an imaging apparatus for an endoscope according to claim 13,
wherein injecting the resin into the outer frame includes injecting a UV-curing resin into the outer frame, and
curing the resin includes curing the UV-curing resin by irradiating the UV-curing resin with UV.
